(19) United States
(12) Patent Application Publication
(10) Pub. No.: US 2016/0102052 A1
(43) Pub. Date: Apr. 14, 2016

(54) Use of Cysteamine in Treating Infections caused by Yeasts/Moulds

(71) Applicant: Novabiotics Limited, Craibstone (GB)

(72) Inventors: Deborah O'NEIL, Craibstone (GB); Vanessa DUNCAN, Craibstone (GB)

(73) Assignee: Novabiotics Limited, Craibstone (GB)

(21) Appl. No.: 14/860,979

(22) Filed: Sep. 22, 2015

Related U.S. Application Data
(60) Provisional application No. 62/053,523, filed on Sep. 22, 2014.

(51) Int. Cl. C07C 323/25; A61K 45/06; A61K 31/7048; A61K 31/496; A61K 38/12; A61K 31/145; A61K 31/4196

(52) U.S. Cl. CPC C07C 323/25; A61K 31/145; A61K 45/06; A61K 31/4196; A61K 31/496; A61K 38/12; A61K 31/7048

(57) ABSTRACT

The present invention relates to compositions comprising cysteamine or a derivative thereof for use in treating infections caused by yeasts or moulds.

### CROSS-REFERENCE TO RELATED APPLICATIONS

The present application claims the benefit of U.S. provisional application No. 62/053,523 filed on Sep. 22, 2014, which is incorporated herein by reference in its entirety for all purposes.

### FIELD OF THE INVENTION

The present invention relates to the use of cysteamine and derivatives thereof in the treatment and/or prevention of infection caused by yeasts and/or moulds.

### BACKGROUND TO THE INVENTION

The frequency of invasive fungal infections has continued to increase over the past two decades, both in the general population and in immunosuppressed patients with the vast majority of infections caused by Aspergillus and Candida species (Pasqualotto, A. C., and Denning, D. W. (2005) Diagnosis of Invasive Fungal Infections—Current Limitations of Classical and New Diagnostic Methods. Euro Oncol Rev). These infections carry high mortality rates and place significant burdens on health care systems. There remains an urgent need for more effective and safe therapeutic agents to treat and prevent infections by yeasts and mould (including e.g. Candida spp. or Aspergillus spp.).

### STATEMENTS OF THE INVENTION

According to a first aspect of the invention, there is provided cysteamine or a derivative thereof (or a composition comprising cysteamine and/or a derivative thereof) for use in the treatment or prevention of an infection caused by yeasts and/or moulds.

The present invention is predicated on the surprising finding that cysteamine and/or derivatives thereof have particular utility in the treatment or prevention of yeast and mould infections, suitably fungal infections such as Aspergillus and/or Candida infections for example.

Suitably, the infection may be a fungal infection or a disease caused by a fungal infection such as e.g., a Candida infection and/or an Aspergillus infection. Suitably, the infection may be caused by yeast and/or moulds. Suitably, the infection may an infection by one or more of the group consisting of: Candida spp., (e.g. C. albicans), Epidermophyton spp., Exophiala spp., Microsporum spp., Trichophyton spp., (e.g T. rubrum and T. interdigitale), Tinea spp., Aspergillus spp., Blastomyces spp., Blastoschizomyces spp., Coccidioides spp., Cryptococcus spp. (e.g. Cryptococcus neoformans), Histoplasma spp., Paracoccidiomyces spp., Sporotrix spp., Absidia spp., Cladophialophora spp., Fonsecaea spp., Phialophora spp., Lacazia spp., Arthrographis spp., Acremonium spp., Actinomadura spp., Apophysomyces spp., Emmonsia spp., Basidiobolus spp., Beauveria spp., Chrysosporium spp., Conidiobolus spp., Cunninghamella spp., Fusarium spp., Geotrichum spp., Graphium spp., Leptosphaeria spp., Malassezia spp. (e.g Malassezia furfur), Mucor spp., Neotestudina spp., Nocardia spp., Nocardiopsis spp., Paecilomyces spp., Phoma spp., Piedraia spp., Pneumocystis spp., Pseudallescheria spp., Pyrenochaeta spp., Rhizomucor spp., Rhizopus spp., Rhodotorula spp., Saccharomyces spp., Scedosporium spp., Scopulariopsis spp., Sporobolomyces spp., Syncephalastrum spp., Trichoderma spp., Trichosporon spp., Ulocladium spp., Ustilago spp., Verticillium spp., Wangiella spp.

Suitably, the infection may be caused by a Candida spp. (e.g. Candida albicans).

Suitably, the infection may be caused by Aspergillus spp. (e.g. Aspergillus fumigatus).

Suitably, the infection may be caused by Exophiala spp. (e.g. Exophiala dermatitides).

Suitably, the infection may be caused by a Cyptococcus spp.

In a further aspect, the present invention provides cysteamine or a derivative thereof or a pharmaceutical composition comprising cysteamine and/or a derivative thereof for use in the prevention or treatment of any one or more of the group consisting of: candidiasis (including OPC), cystic fibrosis, aspergillosis (including bronchopulmonary aspergillosis, chronic pulmonary aspergillosis and aspergillomata), athlete's foot; basidiodiabolomycosis; blastomycosis; coccidioidomycosis cryptoccocis; Chronic obstructive pulmonary disease (COPD); basal meningitis; dermatophytosis; onchomycosis; dermatophytids; endothrix; exothrix; fungal meningitis, fungemia, heaves; histoplasmosis, mycosis, myrinogmycosis, paracoccidioidomycosis, penicilliosis, piedra, pneumocytosis pneumonia, sporptrichosis, tinea, zeospora and zygomycosis. COPD encompasses lung diseases including chronic bronchitis, emphysema and chronic obstructive airways disease.

Suitably, the composition may be a pharmaceutical composition comprising a pharmaceutically acceptable carrier, excipient or diluent.

Suitably, the composition may further comprise an antibiotic or antifungal. Suitably, the antibiotic may be selected from the group consisting of: Tobramycin, Colistin, Gentamicin or Ciprofloxacin. Suitably, the antibiotic may be tobramycin. The antifungal may one or more of Fluconazole, Itraconazole, Caspofungin and Amphotericin B.

Preferably, the composition further comprises an antifungal. Preferably still the composition is for use in treating an infection caused by Exophiala spp.

Suitably, the cysteamine may be used in combination a modified peptide.

As used herein the term “modified peptide” refers to a peptide comprising from 3 to 50 alpha, D and/or L amino acids wherein the amino acids are predominantly arginine and wherein the peptide, optionally further comprises a modification which is selected from one or more of the group consisting of:
1) Incorporation of a histidine tag;
2) lipidation; and
3) pegylation

Suitably, the histidine tag may comprise at least two histidine residues.

In an additional or alternative aspect, the optional modified peptide of the present invention may be a lipidated peptide such that a fatty acid is conjugated to the peptide.

Suitably, the fatty acid may be a C₂ to C₂₀ fatty acid. Preferably, the fatty acid may be C₃ to C₁₄.

In another additional or alternative aspect, the optional modified peptide of the present invention may be PEGylated.

Suitably, the composition of the present invention may further comprise a peptide comprising from 3-500 amino acids wherein the amino acids are predominantly arginine.

In a further aspect the present invention provides a method of treating or preventing an infection caused by yeasts or moulds in a subject comprising administering a pharmaceutically effective amount of a composition comprising cysteamine or a derivative thereof.

Suitably, the infection may be caused by one or more of the group consisting of: Candida spp., (e.g. C. albicans), Aspergillus spp., Epidermophyton spp., Exophiala spp., Microsporum spp., Trichophyton spp., (e.g T. rubrum and T. interdigitale), Tinea spp., Blastomyces spp., Blastoschizomyces spp., Coccidioides spp., Cryptococcus spp. (e.g. Cryptococcus neoformans), Histoplasma spp., Paracoccidiomyces spp., Sporotrix spp., Absidia spp., Cladophialophora spp., Fonsecaea spp., Phialophora spp., Lacazia spp., Arthrographis spp., Acremonium spp., Actinomadura spp., Apophysomyces spp., Emmonsia spp., Basidiobolus spp., Beauveria spp., Chrysosporium spp., Conidiobolus spp., Cunninghamella spp., Fusarium spp., Geotrichum spp., Graphium spp., Leptosphaeria spp., Malassezia spp. (e.g Malassezia furfur), Mucor spp., Neotestudina spp., Nocardia spp., Nocardiopsis spp., Paecilomyces spp., Phoma spp., Piedraia spp., Pneumocystis spp., Pseudallescheria spp., Pyrenochaeta spp., Rhizomucor spp., Rhizopus spp., Rhodotorula spp., Saccharomyces spp., Scedosporium spp., Scopulariopsis spp., Sporobolomyces spp., Syncephalastrum spp., Trichoderma spp., Trichosporon spp., Ulocladium spp., Ustilago spp., Verticillium spp., Wangiella spp.

Suitably, the infection may be caused by a Candida spp. or Aspergillus spp.

Suitably, the infection may be caused by Exophiala spp.

In another aspect, the present invention provides a method of preventing or treating any one or more of the group consisting of: candidiasis (including OPC), cystic fibrosis, aspergillosis (including bronchopulmonary aspergillosis, chronic pulmonary aspergillosis and aspergillomata), athlete's foot; basidiodiabolomycosis; blastomycosis; coccidioidomycosis COPD; cryptoccocis; basal meningitis; dermatophytosis; onchomycosis; dermatophytids; endothrix; exothrix; fungal meningitis, fungemia, heaves; histoplasmosis, mycosis, myrinogmycosis, paracoccidioidomycosis, penicilliosis, piedra, pneumocytosis pneumonia, sporptrichosis, tinea, zeospora and zygomycosis in a patient comprising administering a composition comprising a pharmaceutically effective amount of cysteamine or a derivative thereof to a patient.

Suitably compositions of the present invention may be pharmaceutical compositions comprising a pharmaceutically acceptable carrier, excipient or diluent

Suitably, the composition of the present invention may comprise an antibiotic, such as one or more selected from of the group consisting of: Tobramycin, Colistin, Gentamicin or Ciprofloxacin.

Suitably, the composition may comprise a peptide comprising from 3 to 50 alpha, D and/or L amino acids wherein the amino acids are predominantly arginine and wherein the peptide, optionally further comprises a modification which is selected from one or more of the group consisting of:
1) Incorporation of a histidine tag;
2) lipidation; and
3) pegylation

Suitably, the composition may comprise a peptide comprising from 3-500 amino acids wherein the amino acids are predominantly arginine.

### DETAILED DESCRIPTION

The present invention provides cysteamine or a derivative thereof (or a composition comprising cysteamine and/or a derivative thereof) for use in the treatment or prevention of an infection caused by yeasts and/or moulds.

The term “composition comprising cysteamine and/or a derivative thereof” encompasses the use of cysteamine and/or a derivative thereof optionally in combination with other undefined ingredients. The term “composition comprising” where used may optionally be substituted with “consisting essentially of” or “consisting”.

Examples of cysteamine derivatives include: 2-methylthio ethylamine (cinnamate), 2-methyl thio ethylurea, N-(2-methylthio ethyl)p-acetamido benzamide, 2-aminoethanethiol, N-(2-methylthio ethyl)p-acetamido benzenesulfonamide, N-(2-propylthioethyl)-p-methoxy benzamide, N-(butylthio ethyl)nicotinamide, N-(2-dodecylthio ethyl)p-butoxybenzamide, N-(2-methylthio ethyl)p-toluenesulfonamide, N-(2-isopropylthio ethyl)propionamide, N-(2-octylthio ethyl)acetamide, N-(2-butylthio ethyl)methanesulfonamide, N-(2-isopentylthio ethyl)butane, bis 1,4-(2-acetamido ethylthio), 2,3-butanediol, 2-hexadecylthio ethylamine hydrochloride, 2-allylthio ethylamine malate, 9-octadecene 2-ylthio ethylamine hydrochloride, 2-dodecylthio ethylamine hydrochloride, 2-isopentylthio ethylamine mandelate, 2-octadecylthio ethylamine salicylate, 2-.beta.-hydroxyethyl thio ethylurea, 2-.beta.-hydroxy ethylthio ethylamine hydrochloride, 2-(2,3-dihydroxy propylthio)ethylamine p-toluenesulfonate, 2-(2-hydroxy propylthio)ethylamine oxalate, N-(2-methylthio ethyl)phenylacetamide, 2-(2,2-dimethoxy ethylthio)ethylamine hydrochloride, 2-(2,2-dimethoxy ethylthio)ethylamine undecylenate, 2-(2,2-diethoxy ethylthio)ethylamine undecylenate, 2-(2,2-diethoxy ethylthio)ethylamine acetate,

2-undecenylthio ethylamine, 2-.beta.-ureidoethylthio ethylamine hydrochloride, 2-.beta.-acetamidoethylthio ethylamine tropate, 2,2′-thio diethylamine fumarate, 2,2′-thio diethylurea, 3-.beta.-aminoethylthio propylamine hydrochloride, S-.beta.-ureidoethyl thiocarbamate,
2-ethoxycarbonylthio ethylamine hydrochloride, 2-dimethylamino carbonylthio ethylamine sulfate, 2-butoxycarbonyl methylthio ethylurea, 2-ethyloxycarbonylmethylthio ethylamine hydrochloride, 6-.beta.-aminoethylthio hexanoate of methyl hydrochloride, 5-.beta.-aminoethylthio pentanoic acid, 2-phenylthio ethylamine dihydrogen phosphate, 2-p-t-butylphenylthio ethylamine trichloracetate, 2-p-methoxyphenylthio ethylamine ditartrate, 2-tolylthio ethylamine hydrobromide, 2-(1-biphenyl thio)ethylamine hydrochloride, 2-N-pentachlorophenylthio ethyl acetamide, 2-benzylthio ethylamine malate, 2-benzylthio ethylamine nicotinate, 2-benzylthio 2-methyl propylamine hydrochloride, 2-benzylthio propylamine lactate, N-(2-benzylthio ethyl)nicotinamide hydrochloride, N-(2-benzylthio ethyl)10-undecene amide, N-(2-benzylthio ethyl)hexadecanamide, S-.beta.-aminoethyl mercaptobutyric acid, N-(2-benzylthio ethyl)formamide, N-(2-benzylthio ethyl)phenylacetamide, N-[2-(2,6-dimethyl phenyl)ethyl]hexanamide, 2-o-aminophenylthio ethylamine succinate, N-(2-benzylthio ethyl)glutamine, S-.beta.-aminoethyl mercapto acetic acid (3-S-.beta.-aminoethyl)mercapto propionic acid, (3-S-.gamma.-amino propyl)mercapto acetic acid, S(2-p-methoxybenzamido ethyl)mercapto 2-(2-naphtyl methylthio)ethylamine hydrochloride, 2-(2-naphtyl methylthio)ethylamine disuccinate, (2-thenyl)2-thio ethylamine hydrobromide, 2-N-acetyl(2-thenylthio-ethylamine, 2-o-chlorobenzylthio ethylamine hydrochloride, 2-p-chlorobenzylthio ethylamine glycolate, 2-o-fluorobenzylthio ethylamine hydrochloride, 2-furfurylthio ethylamine hydrochloride, 2-tetrahydrofurfurylthio ethylamine p-amino-benzoate, 2-.beta.-phenylethylthio ethylamine glutamate, 2-diphenylmethylthio ethylamine hydrochloride, 2-triphenyl methylthio ethylamine hydrochloride hemihydrate, 2-(2-pyridyl ethylthio)ethylamine hydrochloride, 2-(2-p-toluene sulfonamido ethylthio)pyridine N-oxide, 2-.beta.-aminoethylthiomethyl pyridine N-oxide dihydrochloride, 2-.beta.-aminoethylthio pyridine N-oxide hydrochloride, 2,4-dichloro 2-benzylthio ethylamine aspartate, N-[2-(3,4-dichloro benzylthio)ethyl]butyramide, N-[2-(2,6-dichloro benzylthio)ethyl]dodecanamide, N-[2-(3,5-dichloro benzylthio)ethyl]trifluoroacetamide hydrochloride, 2-p-ethoxybenzylthio ethylamine hydrochloride, N-[2-m-fluorobenzylthio ethyl]chloroacetamide, 2-p-bromobenzylthio ethylamine succinate, 2-(3,4-dimethoxy benzylthio)ethylamine malate, 2-(3,4-methylenedioxy benzylthio)ethylamine hydrochloride, 2-(2,4-dichloro cetylthio)ethylamine, 2 (3,4,5-trimethoxy benzylthio)ethylamine hydrocinnamate, 2-p-methoxy benzylthio ethylamine salicylate, 2-o-methylbenzylthio ethylamine phenylacetate, N-[2-p-dimethylaminobenzylthio ethyl]methane-sulfonamide, 2-p-phenoxybenzylthio ethylamine hydrochloride, 2-.beta.-aminoethylthio pyridine hydrochloride,
2-benzylthio ethylamine citrate, N-[2-benzylthio ethyl]2,4-dihydroxy 3,3-dimethyl butyramide, N-(2-benzylthio ethyl) 6,8-dihydroxy 7,7-dimethyl 5-oxo 4-aza octanamide, N-[2-(2-pyridyl thio)ethyl]propionamide, 2-(2-pyridyl methylthio)ethylamine dihydrochloride, 2-benzylthio ethyl amine pantothenate, S-(.beta.-acetamidoethyl)mercapto acetate of beta.-morpholinoethyl, S-(.beta.-phenylacetamidoethyl)mercaptoacetate N′-methyl 2-piperazino ethyl, S-(.beta.-ureidoethyl)mercaptoacetate of beta.-pyrrolidino-ethy, S-(.beta.-trifluoroacetamidoethyl)-.beta.mercapto-propionate of .beta.-dimethyl amino ethyl, 2-p-nitrobenzylthio ethylamine crotonate, 2-.beta.-morpholinocarbonyl ethylthio ethylamine hydrochloride, N,N-di(hydroxyethyl)S-(.beta.-benzamido-ethyl)mercapto-acetamido, N[2-N′-methyl piperazino carbonylthio ethyl]acetamide, 2-(1-naphthyl thio)ethylamine hydrochloride, N-(3-.beta.-ureidoethylthio propyl)succinamic acid, 3-allylthio propylamine, 3-(2,2′-dimethoxy ethylthio)propylamine, 3-(2,2′-dimethoxy ethylthio)propylamine sulfate, S-.beta.-aminoethylmercapto acetic acid, the hydrochloride of S-.beta.-aminoethyl mercapto acetic acid, N-(2-benzylthioethyl)acetamide, N-(2-benzylthioethyl)propionamide, N-(2-benzylthioethyl)butyramide, N-(2-benzylthioethyl)methanesulfonamide, N-(2-benzylthioethyl)ethanesulfonamide, N-(2-benzylthioethyl-propanesulfonamide, N-(2-benzylthioethyl)butanesulfonamide, S-(2-p-acetamidobenzenesulfonamido ethyl)mercapto acetic acid, S-(2-p-acetamidobenzamido ethyl)mercapto acetic acid, N-(2-thenylthioethyl)acetamide, 2-benzylthio propylamine, 2-benzylthio 2-methyl propylamine, 2-(2-p-toluenesulfonamido ethylthio)pyridine N-oxide, S-(2-p-butoxybenzamidoethyl)mercapto acetic acid, 2-t-butylthio ethylamine hydrochloride, 2-methoxy carbonyl methylthio ethylamine hydrochloride, 2-ethoxycarbonylmethylthio ethylamine hydrochloride, 2-propoxycarbonylmethyl thio ethylamine hydrochloride, 2-butoxycarbonylmethylthio ethylamine hydrochloride, 2,2′-thio diethylamine dihydrochloride, 3-(2-aminoethylthio)alanine hydrochloride, 2-benzylthio ethylammonium diacid phosphate, 2-methylthio ethylamine, N-(methylthioethyl)p-acetamidobenzamide, N-(2-methylthioethyl)nicotinamide, N-(2-methylthioethyl)benzamide, N-(2-methylthioethyl)p-butoxybenzamide, N-(2-methylthioethyl)butyramide, N-(2-methylthioethyl)propionamide, N-(2-methylthioethyl)acetamide, N-(2-methylthioethyl)butanesulfonamide, N-(2-octylthioethyl)methanesulfonamide, 2-cetylthio ethylamine hydrochloride, 2-(2-hydroxyethylthio)ethylamine hydrochloride, 2-methylthio ethylamine phenylacetate and 2-methylthio ethylamine undecylenate

### Optional Modified Peptide

The compositions of the present invention may further comprise a modified peptide comprising from 3 to 50 D and/or L amino acids wherein the amino acids are predominantly arginine and wherein the peptide comprises a modification which is selected from one or more of the group consisting of:
1) Incorporation of a histidine tag;
2) lipidation; and
3) pegylation

### Incorporation of a Histidine Tag

In one aspect, the modified peptide when used preferably comprises a histidine tag at either the N terminus or C terminus. Advantageously, the presence of a histidine may enhance the effectiveness of the peptide against fungal infections such as Candida. This is extremely unexpected given that the cationic charge may not be significantly changed at such a pH range when compared to an equivalent peptide without the presence of a histidine tag.

Suitably, the histidine tag may comprise at least two histidine residues. Preferably, the number of histidine residues may be up to 10. For example the histidine tag may consist of 1 to 10 histidine residues, preferably 2 to 6. In one embodiment, the histidine tag may consist of two histidine residues

Advantageously, the presence of a histidine tag may be particularly useful for treating fungal infections of the mouth such as oropharyngeal candidiasis.

The oral cavity has a pH between 5.5 and 7 in disease states whereas the normal pH of the mouth of a healthy oral cavity is around pH 7 when not feeding. However, pH influences the charge of AMPs. Furthermore, secreted saliva also contains proteases that aid the breakdown of peptides.

The present inventors have surprisingly found that peptide of the invention modified to comprise a histidine tag are particularly adept at overcoming the pH and protease challenges associated which oral administration.

Accordingly, modified peptides comprising a histidine tag may be comprised in pharmaceutical formulations adapted for oral administration.

Suitably, the peptide used in the pharmaceutical compositions of the present invention, method of treatment or prevention of the present invention and second medical uses of the present invention may comprises a histidine tag when the route of administration or intended route of administration is oral administration.

Preferably, the pH of the pharmaceutical compositions of the present invention is in the region of pH 5.5 to 6.5.

### Lipidation

In one aspect, the modified peptides when used in the composition of the present invention are lipidated. For example, a lipid may be conjugated to a peptide comprising from 3 to 50 D and/or L amino acids wherein the amino acids are predominantly arginine.

The present invention has surprisingly found that lipidation of the peptides can advantageously broaden the spectrum of activity of the peptides against microbes and/or enhance the activity of the peptides against some microbial infections.

Suitably, lipidated peptides of the present invention may be used in the treatment or prevention of yeast and mould infections (preferably such as Candida and/or Aspergillus infections, preferably Aspergillus infections). It has been surprisingly found that lipidation of the peptides claimed can confer potent activity on such lipidated peptides.

Accordingly, the modified peptides of the present invention may comprise a lipid which may be at either the C terminus, N terminus or flanked with amino acid residues.

Suitably the peptides of the present invention may comprise a C₃ to C₂₀ fatty acid, preferably a C₄ to C₁₄ fatty acid, preferably a C₈ to C₁₄ fatty acid, preferably a C₁₂ fatty acid.

Suitably the modified peptides of the present invention may comprise 3 to 50 amino acids and a C₃ to C₂₀ fatty acid, preferably a C₄ to C₁₄ fatty acid, preferably a C₈ to C₁₄ fatty acid, preferably a C₁₂ fatty acid. Preferably, the modified peptides of the present invention may comprise 6 to 50 amino acids and a C₃ to C₂₀ fatty acid, preferably a C₄ to C₁₄ fatty acid, preferably a C₈ to C₁₄ fatty acid, preferably a C₁₂ fatty acid.

In one aspect, the fatty acid may be flanked on either side by amino acid residues. It has surprisingly been found that the flanking of the fatty acid can lead to a reduction in haemolytic activity.

In another aspect, the fatty acid may be located on the terminus of the peptide. It has surprisingly been found that this may increase the antimicrobial effects of the peptide in terms of lower MIC.

In one preferable embodiment the fatty acid is a C₁₂ fatty acid. Advantageously, this length of fatty acids exhibits both good antimicrobial effects and additionally has low cytotoxicity and haemolytic activity.

### PEGylated Peptides

In one aspect the modified peptide of the present invention is a PEGylated peptide.

Advantageously, such PEGylated peptides have enhanced stability whilst still providing antimicrobial effects.

Suitably, the size of the PEG component may be approx. 300 Da to approx. 40 KDa

### Amino Acid Residues

The peptide if used in the composition of the present invention may comprise from 3 to 50 (preferably contiguous) amino acids.

Suitably the peptide may comprise at least 3 or at least 4 or at least 5 or at least 6 or at least 7 or at least 8 or at least 9 or at least 10 or at least 12 or at least 15 or at least 20 or at least 25 or at least 30 or at least 35 or at least 40 or at least 45 amino acids.

Suitably, the peptide may comprise less than 50 or less than 45 or less than 40 or less than 35 or less than 30 or less than 25 or less than 0 or less than 15 amino acids.

In one aspect the number of amino acid residues referred to in the ranges above does not include the histidine tag residues. Thus, in one aspect, histidine residues at either end of the peptide are discounted when determining the numbering of amino acids in the modified peptide. In another aspect, all amino acid residues are counted including those making up a histidine tag.

In a preferred aspect of the invention the peptide comprises 3 to 20 (preferably contiguous) amino acids, for example 3 to 16 amino acids. Preferably still the peptide comprises 5 to 14 amino acids. In some aspects, the peptide may comprise 12 (preferably contiguous) amino acids.

As known to the skilled person, amino acids can be placed into different classes depending primarily upon the chemical and physical properties of the amino acid side chain. For example, some amino acids are generally considered to be hydrophilic or polar amino acids and others are considered to be hydrophobic or non-polar amino acids. Hydrophobic amino acid may be selected from the group of hydrophobic amino acids consisting of glycine, leucine, phenylalanine, proline, alanine, tryptophan, valine, isoleucine, methionine, tyrosine and threonine; cationic amino acids may be selected from the group consisting of ornithine, histidine, arginine and lysine. As used herein, the terms “hydrophobic” and “cationic” may refer to amino acids having a hydrophobicity that is greater than or equal to −1.10 and/or a net charge that is greater than or equal to 0 as described in Fauchere and Pliska Eur. J. Med Chem. 10:39 (1983). A hydrophobic or non-polar amino acid may also refer to an amino acid having a side chain that is uncharged at physiological pH, is not polar and that is generally repelled by aqueous solution. The amino acids may be naturally occurring or synthetic.

Suitably, the arginine residue is the predominant amino acid in the peptide. Suitably, at least 50% of the amino acid residues are arginine residues, preferably at least 60% or at least 70% or at least 80% of the amino acids in the peptide are arginine. Preferably, at least 90% are arginine residues. In some embodiments all the amino acids in the peptide are arginine residues (optionally with the exception of a histidine tag).

Suitably, the peptide may comprise amino acids other than arginine is non-predominant amounts. For example, histidine, ornithine and lysine could be used.

Suitably, 3 to 50 (preferably contiguous) D and/or L amino acids consist of arginine or a combination of arginine and lysine residues except for 0, 1, or 2 substitutions to an amino acid residues other than arginine or lysine. Preferably, such substitutions (if present) are with another cationic amino acids selected from the group consisting of histidine, ornithine and lysine. Preferably the substations are with lysine.

Suitably, the peptide may be substituted with 0, 1, 2, 3, 4, 5, 6, 7 or 8 substitutions provided that the arginine make up at least 60%, preferably at least 75% of the peptide.

Preferably, the amino acids are L-amino acids.

In a preferred aspect of the invention, at least 90%, for example at least 95% such as 97-99% or even 100%, of the amino acids in the peptide are L-amino acids.

The invention also includes known isomers (structural, stereo-, conformational & configurational), peptidomimetics, structural analogues of the above amino acids, and those modified either naturally (e.g. post-translational modification) or chemically, including, but not exclusively, phosphorylation, glycosylation, sulfonylation and/or hydroxylation.

In general, the peptide of the invention does not include the amino acids aspartic acid, glutamic acid, asparagine, glutamine or serine, but certain peptides of the invention may have activity even though these amino acids are present.

One or more of the residues of the peptide can be exchanged for another to alter, enhance or preserve the biological activity of the peptide. Such a variant can have, for example, at least about 10% of the biological activity of the corresponding non-variant peptide. Conservative amino acids are often utilised, i.e. substitutions of amino acids with similar chemical and physical properties as described above. Hence, for example, conservative amino acid substitutions may involve exchanging lysine for arginine, ornithine or histidine; or exchanging arginine for lysine or isoleucine, ornithine for histidine; or exchanging one hydrophobic amino acid for another. After the substitutions are introduced, the variants are screened for biological activity.

### Peptides

The term “peptide” as used herein means, in general terms, a plurality of amino acid residues joined together by peptide bonds. It is used interchangeably and means the same as polypeptide and protein.

The term “modified peptide” refers to a peptide comprising 3 to 50 amino acid residues predominantly arginine further comprising: a histidine tag; and/or a fatty acid and/or a pegylated peptide. Suitably, the modified peptides of the present invention, may be linear peptides.

Preferably, the modified peptides of the present invention may consist of:
1) 3 to 50 amino acid residues predominantly arginine and a histidine tag;
2) 3 to 50 amino acid residues predominantly arginine and one or more fatty acids;
3) 3 to 50 amino acid residues predominantly arginine, a histidine tag and one or more fatty acids;
4) a PEGylated peptide of 3 to 50 amino acid residues predominantly arginine and a histidine tag;
5) a PEGylated peptide of 3 to 50 amino acid residues predominantly arginine and one or more fatty acids; or
6) a PEGylated peptide of 3 to 50 amino acid residues predominantly arginine, a histidine tag and one or more fatty acids.

The peptides may generally be synthetic peptides. The peptides may be isolated, purified peptides or variants thereof, which can be synthesised in vitro, for example, by a solid phase peptide synthetic method, by enzyme catalysed peptide synthesis or with the aid of recombinant DNA technology.

To identify active peptides that have little or no undesired toxicity for mammalian cells, individual peptides, or libraries of peptides, can be made and the individual peptides or peptides from those libraries can be screened for antimicrobial activity and toxicity, including, but not limited to, antifungal, antibacterial, antiviral, antiprotozoal, anti-parasitic activity and toxicity.

The peptides of the invention can exist in different forms, such as free acids, free bases, esters and other prodrugs, salts and tautomers, for example, and the invention includes all variant forms of the compounds.

Thus, the invention encompasses the salt or pro-drug of a peptide or peptide variant of the invention.

### Administration

The composition of the invention may be administered in the form of a pharmaceutically acceptable salt. The pharmaceutically acceptable salts of the present invention can be synthesized from the parent peptide which contains a basic or acidic moiety by conventional chemical methods. Generally, such salts can be prepared by reacting the free acid or base forms of the peptide with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., US, 1985, p. 1418, the disclosure of which is hereby incorporated by reference; see also Stahl et al, Eds, “Handbook of Pharmaceutical Salts Properties Selection and Use”, Verlag Helvetica Chimica Acta and Wiley-VCH, 2002.

The invention thus includes pharmaceutically-acceptable salts of the composition of the invention wherein the parent compound is modified by making acid or base salts thereof for example the conventional non-toxic salts or the quaternary ammonium salts which are formed, e.g., from inorganic or organic acids or bases. Examples of such acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, oxalate, palmoate, pectinate, persulfate, 3-phenylpropionate, picrate, pivalate, propionate, succinate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glutamine, and salts with amino acids such as arginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

Salts of carboxyl groups of a peptide or peptide variant of the invention may be prepared in the usual manner by contacting the peptide with one or more equivalents of a desired base such as, for example, a metallic hydroxide base, e.g. sodium hydroxide; a metal carbonate or bicarbonate such as, for example, sodium carbonate or bicarbonate; or an amine base such as, for example, triethylamine, triethanolamine and the like.

The invention includes prodrugs for the active pharmaceutical species of the described peptide, for example in which one or more functional groups are protected or derivatised but can be converted in vivo to the functional group, as in the case of esters of carboxylic acids convertible in vivo to the free acid, or in the case of protected amines, to the free amino group. The term “prodrug,” as used herein, represents in particular structures which are rapidly transformed in vivo to the parent structure, for example, by hydrolysis in blood.

A further aspect of the invention provides a pharmaceutical composition comprising a pharmaceutically effective amount of cysteamine and/or a derivative thereof. When a modified peptide is used this may form part of the composition or may be administered separately.

The composition may also include a pharmaceutically acceptable carrier, excipient or diluent. The phrase “pharmaceutically acceptable” is employed herein to refer to those compounds, materials, compositions, and/or dosage forms which are, within the scope of sound medical judgment, suitable for use in contact with the tissues of human beings or, as the case may be, an animal without excessive toxicity, irritation, allergic response, or other problem or complication, commensurate with a reasonable benefit/risk ratio.

In a preferred use according to the invention the fungal pathogen is of the genera Candida spp. or Aspergillus spp. For example the fungal pathogen may be Candida albicans, or Aspergillus fumigatus.

The fungal infection may be a systemic, topical, subcutaneous, cutaneous or mucosal infection. Preferably, the fungal infection may be a systemic or mucosal infection.

The compositions of the invention are potent antifungals for a wide variety of pathogenic yeast and moulds. However, the compositions of the invention may also be useful in the treatment of other conditions including, but not limited to, conditions associated with mucosal infections, for example, cystic fibrosis, gastrointestinal, urogenital, urinary (e.g kidney infection or cystitis) or respiratory infections.

In one embodiment, the compositions of the invention are useful in treating the symptoms of respiratory/lung disease, particularly in cystic fibrosis or COPD.

The term “treatment” relates to the effects of the peptides described herein that in imparting a benefit to patients afflicted with an (infectious) disease, including an improvement in the condition of the patient or delay in disease progression.

In a further aspect, the invention provides a method of treating or preventing a microbial infection in a subject comprising administering to said subject a therapeutically effective amount of a composition according to the invention.

In a preferred method of the invention, the microbial infection is a fungal infection. In the method of the invention the peptide is preferably administered orally.

Mammals, birds and other animals may be treated by the peptides, compositions or methods described herein. Such mammals and birds include humans, dogs, cats and livestock, such as horses, cattle, sheep, goats, chickens and turkeys and the like. Moreover, plants may also be treated by the peptides, compositions or methods of the invention.

Where the subject is an animal, the method of the invention may be applied to nail-like features, including, but not exclusive to, hooves, claws and trotters.

To achieve the desired effect(s), the composition, a variant thereof or a combination thereof, may be administered as single or divided dosages, for example, of at least about 0.01 mg/kg to about 500 to 750 mg/kg, of at least about 0.01 mg/kg to about 300 to 500 mg/kg, at least about 0.1 mg/kg to about 100 to 300 mg/kg or at least about 1 mg/kg to about 50 to 100 mg/kg of body weight or at least about 1 mg/kg to about 20 mg/kg of body weight, although other dosages may provide beneficial results. The amount administered will vary depending on various factors including, but not limited to, the peptide chosen and its clinical effects, the disease, the weight, the physical condition, the health, the age of the mammal, whether prevention or treatment is to be achieved, and if the peptide is chemically modified.

Administration of the therapeutic agents in accordance with the present invention may be in a single dose, in multiple doses, in a continuous or intermittent manner, depending, for example, upon the recipient's physiological condition, whether the purpose of the administration is therapeutic or prophylactic, and other factors known to skilled practitioners. The administration of the peptides of the invention may be essentially continuous over a pre-selected period of time or may be in a series of spaced doses. Both local and systemic administration is contemplated.

To prepare the composition, cysteamine and/or a derivative thereof is synthesized or otherwise obtained, purified as necessary or desired, and then lyophilized and stabilized. The composition can then be adjusted to the appropriate concentration and optionally combined with other agents. The absolute weight of a given peptide included in a unit dose can vary widely. For example, about 0.01 mg to about 2 g or about 0.01 mg to about 500 mg, of at least one peptide of the invention, or a plurality of peptides specific for a particular cell type can be administered. Alternatively, the unit dosage can vary from about 0.01 g to about 50 g, from about 0.01 g to about 35 g, from about 0.1 g to about 25 g, from about 0.5 g to about 12 g, from about 0.5 g to about 8 g, from about 0.5 g to about 4 g, or from about 0.5 g to about 2 g.

Thus, one or more suitable unit dosage forms comprising the therapeutic composition of the invention can be administered by a variety of routes including oral, parenteral (including subcutaneous, intravenous, intramuscular and intraperitoneal), rectal, dermal, transdermal, intrathoracic, intrapulmonary and intranasal (respiratory) routes. The therapeutic peptides may also be formulated in a lipid formulation or for sustained release (for example, using microencapsulation, see WO 94/07529, and U.S. Pat. No. 4,962,091). The formulations may, where appropriate, be conveniently presented in discrete unit dosage forms and may be prepared by any of the methods well-known to the pharmaceutical arts. Such methods may include the step of mixing the therapeutic agent with liquid carriers, solid matrices, semi-solid carriers, finely divided solid carriers or combinations thereof, and then, if necessary, introducing or shaping the product into the desired delivery system.

When the therapeutic compositions of the invention are prepared for oral administration, they are generally combined with a pharmaceutically acceptable carrier, diluent or excipient to form a pharmaceutical formulation, or unit dosage form. For oral administration, the peptides may be present as a powder, a granular formation, a solution, a suspension, an emulsion or in a natural or synthetic polymer or resin for ingestion of the active ingredients from a chewing gum. The active ingredients may also be presented as a bolus, electuary or paste. Orally administered therapeutic compositions of the invention can also be formulated for sustained release, e.g., cysteamine can be coated, micro-encapsulated, or otherwise placed within a sustained delivery device. The total active ingredients in such formulations comprise from 0.1% to 99.9% by weight of the formulation.

Pharmaceutical formulations containing the therapeutic composition of the invention can be prepared by procedures known in the art using well-known and readily available ingredients. For example, the peptide can be formulated with common excipients, diluents, or carriers, and formed into tablets, capsules, solutions, suspensions, powders, aerosols and the like. Examples of excipients, diluents, and carriers that are suitable for such formulations include buffers, as well as fillers and extenders such as starch, cellulose, sugars, mannitol, and silicic derivatives. Binding agents can also be included such as carboxymethyl cellulose, hydroxymethylcellulose, hydroxypropyl methylcellulose and other cellulose derivatives, alginates, gelatine, and polyvinyl-pyrrolidone. Moisturizing agents can be included such as glycerol, disintegrating agents such as calcium carbonate and sodium bicarbonate. Agents for retarding dissolution can also be included such as paraffin. Resorption accelerators such as quaternary ammonium compounds can also be included. Surface active agents such as cetyl alcohol and glycerol monostearate can be included. Adsorptive carriers such as kaolin and bentonite can be added. Lubricants such as talc, calcium and magnesium stearate, and solid polyethyl glycols can also be included. Preservatives may also be added. The compositions of the invention can also contain thickening agents such as cellulose and/or cellulose derivatives. They may also contain gums such as xanthan, guar or carbo gum or gum arabic, or alternatively polyethylene glycols, bentones and montmorillonites, and the like.

For example, tablets or caplets containing the composition of the invention can include buffering agents such as calcium carbonate, magnesium oxide and magnesium carbonate. Suitable buffering agents may also include acetic acid in a salt, citric acid in a salt, boric acid in a salt and phosphoric acid in a salt. Caplets and tablets can also include inactive ingredients such as cellulose, pregelatinized starch, silicon dioxide, hydroxyl propyl methyl cellulose, magnesium stearate, microcrystalline cellulose, starch, talc, titanium dioxide, benzoic acid, citric acid, corn starch, mineral oil, polypropylene glycol, sodium phosphate, zinc stearate, and the like. Hard or soft gelatine capsules containing at least one peptide of the invention can contain inactive ingredients such as gelatine, microcrystalline cellulose, sodium lauryl sulphate, starch, talc, and titanium dioxide, and the like, as well as liquid vehicles such as polyethylene glycols (PEGs) and vegetable oil. Moreover, enteric-coated caplets or tablets containing one or more peptides of the invention are designed to resist disintegration in the stomach and dissolve in the more neutral to alkaline environment of the duodenum.

The therapeutic composition of the invention can also be formulated as elixirs or solutions for convenient oral administration or as solutions appropriate for parenteral administration, for instance by intramuscular, subcutaneous, intraperitoneal or intravenous routes. The pharmaceutical formulations of the invention can also take the form of an aqueous or anhydrous solution or dispersion, or alternatively the form of an emulsion or suspension or salve.

Thus, the therapeutic compositions may be formulated for parenteral administration (e.g. by injection, for example, bolus injection or continuous infusion) and may be presented in unit dose form in ampules, pre-filled syringes, small volume infusion containers or in multi-dose containers. The active ingredients may form suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution for constitution with a suitable vehicle, e.g. sterile, pyrogen-free water before use.

These formulations can contain pharmaceutically acceptable carriers, vehicles and adjuvants that are well-known in the art. It is possible, for example, to prepare solutions using one or more organic solvent(s) that is/are acceptable from the physiological standpoint, chosen, in addition to water, from solvents such as acetone, acetic acid, ethanol, isopropyl alcohol, dimethyl sulphoxide, glycol ethers such as the products sold under the name “Dowanol”, polyglycols and polyethylene glycols, C₁-C₄ alkyl esters of short-chain acids, ethyl or isopropyl lactate, fatty acid triglycerides such as the products marketed under the name “Miglyol”, isopropyl mytrisate, animal, mineral and vegetable oils and polysiloxanes.

Solvents or diluents comprising the peptides of the invention may include acid solutions, dimethylsulphone, N-(2-mercaptopropionyl)glycine, 2-n-nonyl-1,3-dioxolane and ethyl alcohol. Preferably the solvent/diluent is an acidic solvent, for example, acetic acid, citric acid, boric acid, lactic acid, propionic acid, phosphoric acid, benzoic acid, butyric acid, malic acid, malonic acid, oxalic acid, succinic acid or tartaric acid.

Also contemplated are combination products that include one or more peptides of the present invention and one or more other antimicrobial or antifungal agents, for example, polyenes such as amphotericin B, amphotericin B lipid complex (ABCD), liposomal amphotericin B (L-AMB), and liposomal nystatin, azoles and triazoles such as voriconazole, fluconazole, ketoconazole, itraconazole, pozaconazole and the like; glucan synthase inhibitors such as caspofungin, micafungin (FK463), and V-echinocandin (LY303366); griseofulvin; allylamines such as terbinafine; flucytosine or other antifungal agents, including those described herein. In addition, it is contemplated that the peptides might be combined with topical antifungal agents such as ciclopirox olamine, haloprogin, tolnaftate, undecylenate, topical nysatin, amorolfine, butenafine, naftifine, terbinafine, and other topical agents.

Additionally, the compositions may be formulated as sustained release dosage forms and the like. The formulations can be so constituted that they release the active peptide, for example, in a particular part of the intestinal or respiratory tract, possibly over a period of time. Coatings, envelopes, and protective matrices may be made, for example, from polymeric substances, such as polylactide-glycolates, liposomes, microemulsions, microparticles, nanoparticles, or waxes. These coatings, envelopes, and protective matrices are useful to coat indwelling devices, e.g. stents, catheters, peritoneal dialysis tubing, draining devices and the like.

For topical administration, the active agents may be formulated as is known in the art for direct application to a target area. Forms chiefly conditioned for topical application take the form, for example, of creams, milks, gels, powders, dispersion or microemulsions, lotions thickened to a greater or lesser extent, impregnated pads, ointments or sticks, aerosol formulations (e.g. sprays or foams), soaps, detergents, lotions or cakes of soap. Other conventional forms for this purpose include wound dressings, coated bandages or other polymer coverings, ointments, creams, lotions, pastes, jellies, sprays, and aerosols. Thus, the therapeutic peptides of the invention can be delivered via patches or bandages for dermal administration. Alternatively, the composition can be formulated to be part of an adhesive polymer, such as polyacrylate or acrylate/vinyl acetate copolymer. For long-term applications it might be desirable to use microporous and/or breathable backing laminates, so hydration or maceration of the skin can be minimized. The backing layer can be any appropriate thickness that will provide the desired protective and support functions. A suitable thickness will generally be from about 10 microns to about 200 microns.

Topical administration may be in the form of a nail coating or lacquer. For example, the antifungal peptides can be formulated in a solution for topical administration that contains ethyle acetate (NF), isopropyl alcohol (USP), and butyl monoester of poly[methylvinyl ether/maleic acid] in isopropyl alcohol.

Pharmaceutical formulations for topical administration may comprise, for example, a physiologically acceptable buffered saline solution containing between about 0.001 mg/ml and about 100 mg/ml, for example between 0.1 mg/ml and 10 mg/ml, of one or more of the peptides of the present invention specific for the indication or disease to be treated.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The active peptides can also be delivered via iontophoresis, e.g., as disclosed in U.S. Pat. Nos. 4,140,122; 4,383,529; or 4,051,842. The percentage by weight of a therapeutic agent of the invention present in a topical formulation will depend on various factors, but generally will be from 0.01% to 95% of the total weight of the formulation, and typically 0.1-85% by weight.

Drops, such as eye drops or nose drops, may be formulated with one or more of the therapeutic peptides in an aqueous or non-aqueous base also comprising one or more dispersing agents, solubilizing agents or suspending agents. Liquid sprays can be pumped, or are conveniently delivered from pressurized packs. Drops can be delivered via a simple eye dropper-capped bottle, via a plastic bottle adapted to deliver liquid contents drop-wise, or via a specially shaped closure.

The therapeutic peptide may further be formulated for topical administration in the mouth or throat. For example, the active ingredients may be formulated as a lozenge further comprising a flavoured base, usually sucrose and acacia or tragacanth; pastilles comprising the composition in an inert base such as gelatine and glycerine or sucrose and acacia; and mouthwashes comprising the composition of the present invention in a suitable liquid carrier. Alternatively, the active ingredients may be formulated as a film strip or buccal tablet, which may or may not be dissolvable.

Specific non-limiting examples of the carriers and/or diluents that are useful in the pharmaceutical formulations of the present invention include water and physiologically acceptable buffered saline solutions such as phosphate buffered saline solutions pH 7.0-8.0.

The compositions of the invention can also be administered to the respiratory tract. For administration by inhalation or insufflation, the composition may take the form of a dry powder, for example, a powder mix of the therapeutic agent and a suitable powder base such as lactose or starch. Therapeutic peptides of the present invention can also be administered in an aqueous solution when administered in an aerosol or inhaled form. Thus, other aerosol pharmaceutical formulations may comprise, for example, a physiologically acceptable buffered saline solution containing between about 0.001 mg/ml and about 100 mg/ml for example between 0.1 and 100 mg/ml, such as 0.5-50 mg/ml, 0.5-20 mg/ml, 0.5-10 mg/ml, 0.5-5 mg/ml or 1-5 mg/ml of one or more of the peptides of the present invention specific for the indication or disease to be treated.

### Antibiotic/Antifungal Agent

The compositions of the present invention may further comprise an antibiotic. The term “antibiotic” is used to refer to antibacterial agents that may be derived from bacterial sources. Antibiotic agents may be bactericidal and/or bacteriostatic.

Generally the antibiotic agent is of the group consisting of aminoglycosides, ansamycins, carbacephem, carbapenems, cephalosporins (including first, second, third, fourth and fifth generation cephalosporins), lincosamides, macrolides, monobactams, nitrofurans, quinolones, penicillin, sulfonamides, polypeptides and tetracyclins. Alternatively or additionally the antibiotic agent may be effective against mycobacteria.

According to one embodiment, the antibiotic agent may be an aminoglycoside such as Amikacin, Gentamicin, Kanamycin, Neomycin, Netilmicin, Tobramycin or Paromomycin.

According to one embodiment, the antibiotic agent may be an http://en.wikipedia.org/wiki/Ansamycin such as Geldanamycin and Herbimycin

Alternatively the antibiotic agent may be a carbacephem such as Loracarbef.

According to a further embodiment, the antibiotic agent is a carbapenem such as Ertapenem, Doripenem, Imipenem/Cilastatin or Meropenem.

Alternatively the antibiotic agent may be a cephalosporins (first generation) such as Cefadroxil, Cefazolin, Cefalexin, Cefalotin or Cefalothin, or alternatively a Cephalosporins (second generation) such as Cefaclor, Cefamandole, Cefoxitin, Cefprozil or Cefuroxime. Alternatively the antibiotic agent may be a Cephalosporins (third generation) such as Cefixime, Cefdinir, Cefditoren, Cefoperazone, Cefotaxime, Cefpodoxime, Ceftibuten, Ceftizoxime and Ceftriaxone or a Cephalosporins (fourth generation) such as Cefepime and Ceftobiprole.

The antibiotic agent may be a lincosamides such as Clindamycin and Azithromycin, or a macrolide such as Azithromycin, Clarithromycin, Dirithromycin, Erythromycin, Roxithromycin, Troleandomycin, Telithromycin and Spectinomycin.

Alternatively the antibiotic agent may be a monobactams such as Aztreonam, or a nitrofuran such as Furazolidone or Nitrofurantoin.

The antibiotic agent may be a penicillin such as Amoxicillin, Ampicillin, Azlocillin, Carbenicillin, Cloxacillin, Dicloxacillin, Flucloxacillin, Mezlocillin, Nafcillin, Oxacillin, Penicillin G or V, Piperacillin, Temocillin and Ticarcillin.

The antibiotic agent may be a sulfonamide such as Mafenide, Sulfonamidochrysoidine, Sulfacetamide, Sulfadiazine, Silver sulfadiazine, Sulfamethizole, Sulfamethoxazole, Sulfanilimide, Sulfasalazine, Sulfisoxazole, Trimethoprim, and Trimethoprim-Sulfamethoxazole (Co-trimoxazole) (TMP-SMX).

The antibiotic agent may be a quinolone such as Ciprofloxacin, Enoxacin, Gatifloxacin, Levofloxacin, Lomefloxacin, Moxifloxacin, Nalidixic acid, Norfloxacin, Ofloxacin, Trovafloxacin, Grepafloxacin, Sparfloxacin and Temafloxacin.

According to one embodiment, the antibiotic agent may be a polypeptide such as Bacitracin, Colistin and Polymyxin B.

Alternatively, the antibiotic agent may be a tetracycline such as Demeclocycline, Doxycycline, Minocycline and Oxytetracycline

Alternatively or additionally the antibiotic agent may be effective against mycobacteria. In particular the antibiotic agent may be Clofazimine, Lamprene, Dapsone, Capreomycin, Cycloserine, Ethambutol, Ethionamide, Isoniazid, Pyrazinamide, Rifampicin, Rifabutin, Rifapentine or Streptomycin.

Generally the antibiotic agent is active in the treatment or prophylaxis of infections caused by gram-negative or gram-positive bacteria, such as Escherichia coli and Klebsiella particularly Pseudomonas aeruginosa.

The ratio of cysteamine and/or a derivative thereof to antibiotic in the products of the invention may be from 1:10 to 10:1; generally at least 2:1 for example at least 3:1 or 4:1. Alternatively, the ratio of the antibiotic agent to the second agent in the products of the invention may be from 1:100 1:2000, for example from 1:500 to 1:1000. According to one embodiment, the ratio of the antibiotic agent to the second agent is approximately 1:1. Preferably the first antibiotic agent is a non-peptide antibiotic and the second agent is cysteamine and the product contains these components at a ratio from 2:1 up to 4:1. According to a further embodiment the ratio may be approximately 1:1.

The antifungal may selected from the group consisting of Fluconazole, Itraconazole, Caspofungin and Amphotericin B, for example, one or more of Fluconazole, Itraconazole and Caspofungin.

The active agents may be administered simultaneously, sequentially or separately. The active agents may be provided as a combination package. The combination package may contain the product of the invention together with instructions for simultaneous, separate or sequential administration of each of the active agents. For sequential administration, the active agents can be administered in any order.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### FIGURES

FIG. 1 shows the effects of cysteamine (Lynovex, NM001) on Candida albicans 73/025 after 24 hours.

FIG. 2 shows the effects of cysteamine (Lynovex, NM001) on Candida albicans 73/025 after 48 hours.

FIG. 3A-FIG. 3B shows the effect of cysteamine (NM001) on four Aspergillus fumigatus strains (AF20, AF22 and NCPF2939 in FIG. 3A and AF 2002/0066 in FIG. 3B).

FIG. 4A-FIG. 4D show petri dishes with Aspergillus growth isolated from the sputum of 4 patients with cystic fibrosis.

FIG. 5A-FIG. 5B shows the microbial load of the sputum of 10 cystic fibrosis patients at 4 hours (FIG. 5A) and 24 hours (FIG. 5B) of treatment with cysteamine (Lynovex), Tobramycin or a combination thereof.

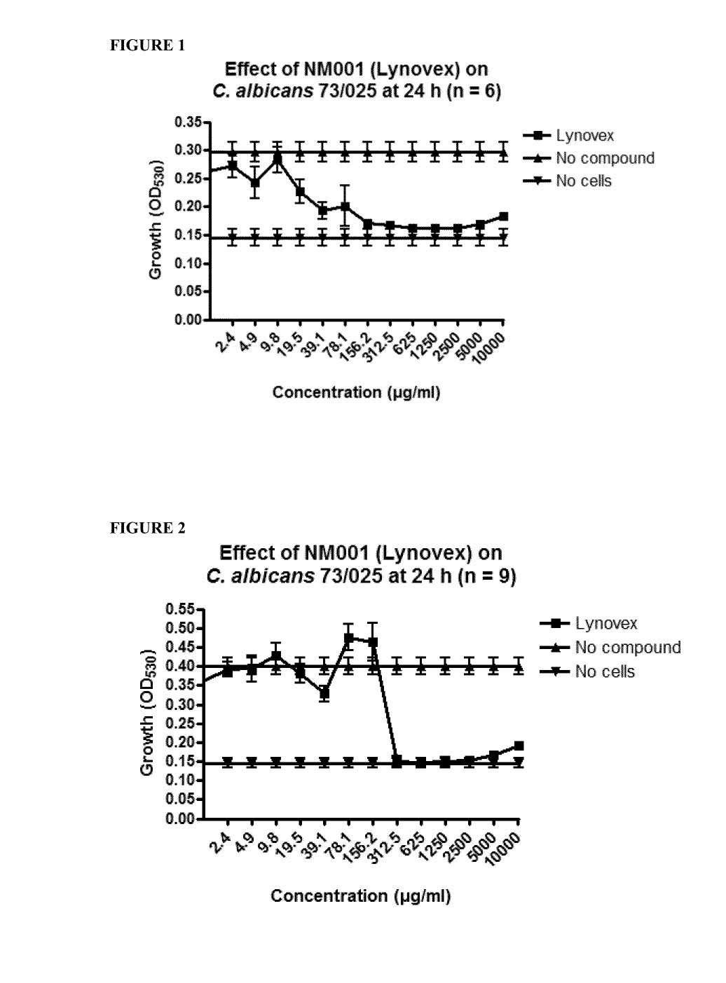

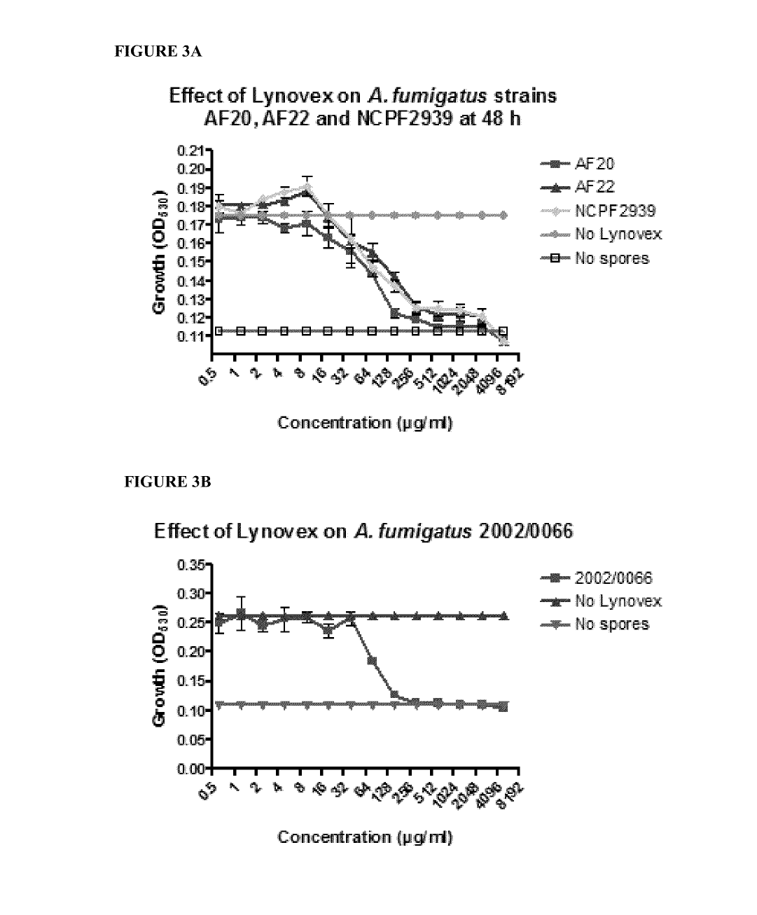

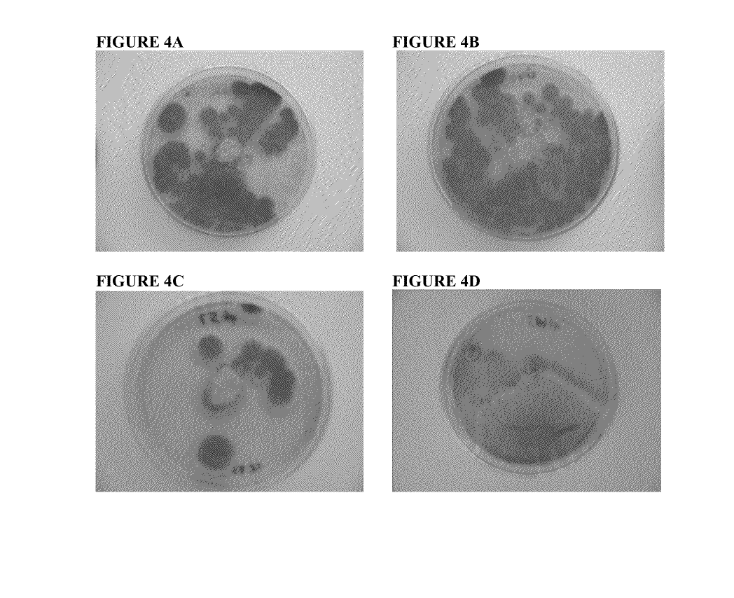

### EXAMPLES

The following Example illustrates the invention.

### Example 1

### Minimum Inhibitory Concentration Test of Cysteamine (Lynovex) (NM001) Against a Vaginal Isolate of C. albicans

### Aim:

The aim of the experiment was to determine the MIC of Lynovex (NM001) against vulvovaginal isolate Candida albicans 73/025. Plates were prepared which contain a serial doubling dilution of Lynovex (NM001) at 2× final concentration. The cultures were then prepared in 2×RPMI-1640, according to the CLSI M27-A3 protocol, and mixed in equal volumes in the plate resulting in 1×RPMI-1640, the concentration of compound mentioned below, and the CLSI standard inoculum for C. albicans. These were incubated at 35° C. and read at 24 h and 48 h on the BioTek plate reader.

### Compounds for Antifungal Testing

### Lynovex (NM001)

### Method:

### Culture Preparation

1. Culture of Candida albicans grown overnight at 30° C.
2. Culture removed from incubator. Add 2 ml sterile dH₂O to the overnight culture and wash the cells from the surface of the agar. Cells placed in fresh bijou tube.
3. Serial doubling dilution carried out and measured against same volume of MacFarland Standard
   a. 100 μl culture added to well A1 of microtitre plate.
   b. 100 μl sterile dH₂O added to wells A2-A11.
   c. 100 μl culture added to well A2 of microtitre plate and mixed well resulting in a 1 in 2 dilution.
   d. 100 μl taken from A2 and added to A3 on plate and mixed well resulting in a 1 in 4 dilution.
   e. This is continued to A11 where 100 μl is removed and discarded.
   f. Well A12 contains 100 μl 0.5 MacFarland standard which had been mixed well.
   g. Plate read at OD₅₃₀ on BioTek
   h. Concentration of cells required will be the one that is the equivalent to the 0.5 MacFarland Standard
4. Dilute cells to the equivalent of the 0.5 MacFarland Standard (for example a 1 in 16 dilution may be required) in sterile dH₂O
5. Culture then further diluted 1 in 50 into water (which is 100 μl culture+4900 μl sterile water).
6. Culture then further diluted 1 in 20 into 2×RPMI-1640 (which is 1 ml culture+19 ml 2×RPMI-1640).

### Plate Preparation

1. The highest final concentration on the plate will be as described in layouts at the end of the protocol. To allow for the addition of the cells this is prepared at 2× final concentration described e.g 20,000 μg/ml is prepared, and so when cells are added this halves to 10,000 μg/ml.
2. Solutions of each of the compounds are prepared in a sterile bijoux giving a final volume of 5 ml (5000 μl).
3. Wells A1-A3, A4-A6, A7-A9 and A10-A12 are prepared by the addition of 100 μl highest concentration of compound to the well (except Clotrimazole plate where the compounds go in A1-3 and A7-9 only).
4. All other wells contain 50 μl sterile dH₂O.
5. Serial doubling dilutions are carried out from A1-A3 to B1-B3 whereby 50 μl is removed from A1-A3 and placed into B1-B3 and mixed well. Then take 50 μl B1-B3 and place in C1-C3 and mix well. Take 50 μl from C1-C3 and add to D1-D3 and mix well. This is repeated down to F1-F3. At F1-F3 50 μl is removed and discarded. G1-G3 and H1-H3 contain water alone and will be untreated and uninoculated controls.
6. The same procedure is repeated for the wells in columns A4-A6, A7-A9 and A10-A12. See plate layout at bottom of page.
   Inoculating Plates with Candida
1. Add 50 μl of the prepared Candida culture to the all wells on the plate except wells H1-H12 (bottom row) which will contain water and 2×RPMI-1640 alone, resulting in an uninoculated control.
2. The plate is read in the BioTek at OD₅₃₀.
3. Plates are then placed in a plastic box which contains either a Petri dish of water or a bed of damp tissue. The box is sealed and placed in the 35° C. The presence of the water in the dish or on the tissue provides and environment of relatively high humidity.
4. Plates are read at 24 h and 48 h and MIC graphs plotted.

### Plate Layout

### Lynovex Plate

| | 1 | 2 | 3 | 4 | 5 | 6 | 7 | 8 | 9 | 10 | 11 | 12 |
|---|---|---|---|---|---|---|---|---|---|---|---|---|
| A | 10,000 | | | 39.0625 | | | | | | | | |
| B | 5,000 | | | 19.53125 | | | | | | | | |
| C | 2,500 | | | 9.765625 | | | | | | | | |
| D | 1250 | | | 4.8828125 | | | | | | | | |
| E | 625 | | | 2.44140625 | | | | | | | | |
| F | 312.5 | | | 1.220703125 | | | | | | | | |
| G | 156.25 | | | 0 | | | | | | | | |
| H | 78.125 | | | No cells | | | | | | | | |

### Results

As can be seen Cysteamine inhibited the growth of Candida albicans.

### Example 2

### Minimum Inhibitory Concentration Test of Lynovex (Cysteamine) (NM001) Against Aspergillus fumigatus

### Aim:

The aim of the experiment was to determine the MIC of Lynovex (NM001) against various Aspergillus fumigatus strains. Microtitre plates were prepared which contained a serial doubling dilution of Lynovex (NM001) at 2× final concentration. The cultures were then prepared in 2×RPMI-1640, according to the CLSI M38-A2 protocol, and mixed in equal volumes in the plate resulting in 1×RPMI-1640, the concentration of compound mentioned below, and the CLSI standard inoculum for A. fumigatus. These were incubated at 37° C. and read at 24 h and 48 h on the BioTek plate reader.

### Compounds for Antifungal Testing

### Lynovex (NM001)

### Method:

### Culture Preparation

1. Cultures of A. fumigatus 2002/0066, AF20, AF22 and NCPF2939 grown at 37° C. on Potato Dextrose Agar (PDA) slopes for >72 h to allow for optimal spore production).
2. Culture removed from incubator and 1 ml sterile 0.85% saline added to the culture on the slope to wash the culture from the surface of the agar. Spores were collected in a fresh tube.
3. Serial doubling dilution of the spores were carried out and measured at an optical density of 530 nm. The cell density was then adjusted to and OD 530 nm of 0.09 to 0.13, as specified in the CLSI document M38A.
4. The spores were then further diluted 1 in 50 into 2×RPMI-1640.
5. The diluted spores were then added in equal volumes to the Lynovex in the microtitre plates (50 μl cells added to 50 μl Lynovex in the microtitre plate).

### Plate Preparation

1. A stock solution of Lynovex was prepared at 10 mg/ml. The plate layout at the end of this protocol is the final concentration in the plate following the addition of the spores, therefore the plate has to be prepared at 2× final concentration to allow the dilution when the cells are added. Therefore the Lynovex prepared in the plate is 10 mg/ml (10,000 μg/ml) and the final concentration in the plate following addition of cells is 5 mg/ml (5,000 μg/ml).
2. 100 μl of Lynovex at 10 mg/ml is added to wells A1-A3 and A7-A9.
3. All other wells contain 50 μl sterile dH₂O.
4. Serial doubling dilutions are carried out from A1-A3 to B1-B3 whereby 50 μl is removed from A1-A3 and placed into B1-B3 and mixed well. Then take 50 μl B1-B3 and place in C1-C3 and mix well. Take 50 μl from C1-C3 and add to D1-D3 and mix well. This is repeated down to H1-H3. At H1-H3 50 μl is removed and added to A4-6 and the dilutions carried down to F4-6. G4-6 and H4-6 contain water alone and will be untreated and uninoculated controls.
5. The same procedure is repeated for the wells in the second half of the plate with Lynovex being added to A7-9 and dilutions being carried out to H7-9 and then continuing from A10-12.
   Inoculating Plates with A. fumigatus
1. Add 50 μl of the prepared A. fumigatus spores to the all wells on the plate except wells H4-6 and H10-H12 which will contain water and 2×RPMI-1640 alone, resulting in an uninoculated control.
2. G4-6 and G10-12 are the inoculated controls and have spores but no Lynovex.
3. The plate is read in the BioTek at OD₅₃₀.
4. Plates are then placed in a plastic box which contains either a Petri dish of water or a bed of damp tissue. The box is sealed and placed in the 37° C. The presence of the water in the dish or on the tissue provides and environment of relatively high humidity.
5. Plates are read at 24 h and 48 h and MIC graphs plotted.

### Plate Layout

### Final Lynovex Plate Following Addition of Spores.

| | 1 | 2 | 3 | 4 | 5 | 6 | 7 | 8 | 9 | 10 | 11 | 12 |
|---|---|---|---|---|---|---|---|---|---|---|---|---|
| A | 5,000 | | | 19.53125 | | | 5,000 | | | 19.53125 | | |
| B | 2,500 | | | 9.765625 | | | 2,500 | | | 9.765625 | | |
| C | 1250 | | | 4.8828125 | | | 1250 | | | 4.8828125 | | |
| D | 625 | | | 2.44140625 | | | 625 | | | 2.44140625 | | |
| E | 312.5 | | | 1.220703125 | | | 312.5 | | | 1.220703125 | | |
| F | 156.25 | | | 0.610365625 | | | 156.25 | | | 0.610365625 | | |
| G | 78.125 | | | 0 | | | 78.125 | | | 0 | | |
| H | 39.0625 | | | No cells | | | 39.0625 | | | No cells | | |

### Example 3

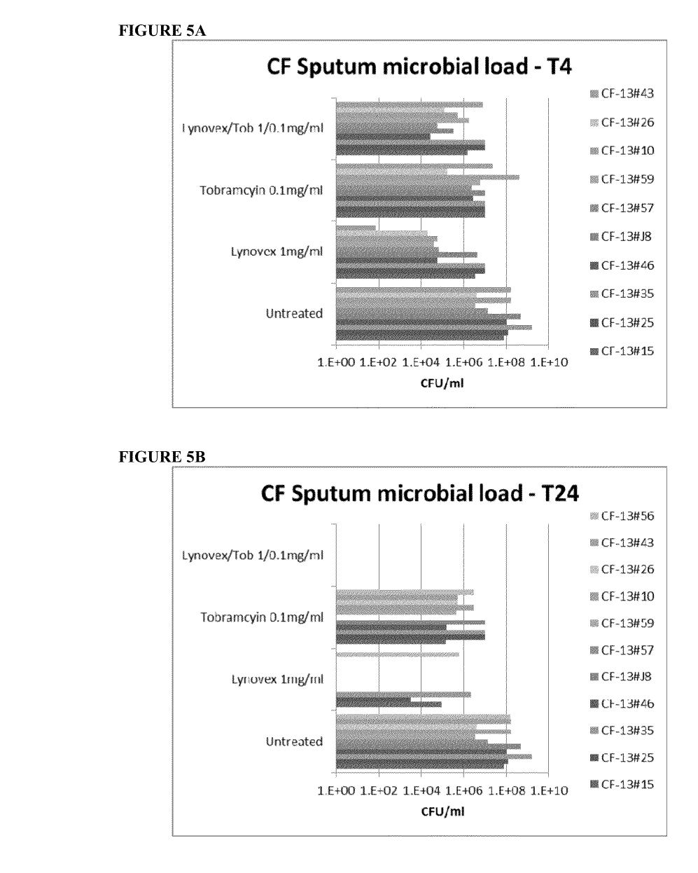

Freshly produced sputum samples were obtained from CF patients and samples of this were exposed to a single dose of 1 mg/ml cysteamine of 1 mg/ml cysteamine and tobramycin (10-100 μg/ml) before being plated out and incubated for 4 h and 24 h before assessment of the resulting number of colony forming units of microbes as compared to those from untreated sputum samples. Results are shown in FIG. 5.

### Example 4

### Minimum Inhibitory Concentration Test of Itraconazole, Fluconazole and Caspofungin Against a Clinical Isolate of E. dermatitidis

### Aim:

The aim of the experiment was to determine the MIC of Itraconazole, Fluconazole and Caspofungin against the clinical isolate Exophiala dermatitidis CA01. Two Candida spp. strains (C. krusei ATCC6258 & C. parapsilosis ATCC22019) and Exophiala jeanselmei reference strain NCPF2377 were also tested. These acted as quality control strains. Flat-bottomed Nunc Plates were prepared containing a serial doubling dilution of antifungals at 2× final concentration. Cultures were then prepared in 2×RPMI-1640, according to the CLSI M38-A2 protocol, and mixed in equal volumes in the plate resulting in 1×RPMI-1640, the concentration of compound mentioned below, and the CLSI standard inoculum. These were incubated at 35° C. and read at 24 h and 48 h (Candida spp.) or 120 h (Exophiala spp.) on a BioTek plate reader at 530 nm.

### Compounds for Antifungal Testing

### Itraconazole

### Fluconazole

### Caspofungin

### Dimethylsulfoxide (DMSO; Vehicle Control)

### Method:

### Culture Preparation:

7. Culture of Candida spp. grown on Sabouraud dextrose agar slope overnight at 30° C.
8. Exophiala cultures grown on potato dextrose agar slope for 6 days at 35° C.
9. Cultures were removed from incubator and 2 ml sterile 0.15M NaCl was added to the overnight culture and spore and hyphal suspensions were harvested from the surface of the agar and transferred to a sterile plastic bijou tube.
   a. Exophiala spp. cultures were allowed to rest for up to 10 min, allowing hyphae to settle to the bottom. The top layers of spores were transferred to a sterile plastic bijou tube.
10. Two-fold, serial dilutions were carried out and optical density measured spectrophotometrically to attain the 0.5 McFarland Standard
   a. 100 μl culture added to A1 of microtitre plate.
   b. 100 μl sterile 0.15M NaCl added to wells A2-A12.
   c. 100 μl culture added to well A2 of microtitre plate and mixed by pipetting. This results in a 1 in 2 dilution.
   d. 100 μl taken from A2 and added to A3 on plate and mixed by pipetting (1 in 4 dilution).
   e. This is continued to A12 where 100 μl is removed and discarded.
   f. This dilution series was repeated in rows B-D for the remaining 3 cultures
   g. 0.5 McFarland Standard added to wells E1-E3
   h. Plate read at OD₅₃₀ on BioTek.
   i. Concentration of cells required will be the one that is the equivalent to the 0.5 McFarland Standard
11. Dilute cells to the equivalent of the 0.5 McFarland Standard (for example a 1 in 32 dilution may be required) in sterile 0.15M NaCl
12. Culture then further diluted 1 in 50 into 2×RPMI-1640 (100 μl culture+4900 μl 2×RPMI-1640).

### Plate Preparation

7. The highest final concentration on the plate will be as described in layouts at the end of the protocol. To allow for the addition of the cells this is prepared at 2× final concentration described e.g. 2000 μg/ml Fluconazole is prepared, and so when cells are added this halves to 1000 μg/ml.
8. Solutions of each of the test compounds are prepared in a sterile plastic bijou tube giving a final volume of 5 ml.
9. Wells A1-A3 and A7-A9 are prepared by the addition of 200 μl highest concentration of compound to the wells.
10. All other wells contain 100 μl sterile dH₂O.
11. Serial dilutions are carried out from A1-A3 to B1-B3 whereby 100 μl is removed from A1-A3 and placed into B1-B3 and mixed well by pipetting. Then take 100 μl B1-B3 and place in C1-C3 and mix well. Take 100 μl from C1-C3 and add to D1-D3 and mix well. This is repeated down to H1-H3. At H1-H3100 μl is removed and added to A4-A6. The dilution series is carried down the plate in the same manner to wells G4-G6 where a final 100 μl is removed and discarded. H4-H6 contain water alone and will be untreated controls.
12. The same procedure is repeated for the wells in columns A7-A9 and A10-A12. See plate layout at bottom of page.
   Inoculating Plates with Candida spp./Exophiala spp.
5. Add 100 μl of the prepared cultures to the all wells on the plate except wells H10-H12 (bottom row) which will contain water and 2×RPMI-1640 alone, resulting in an uninoculated control.
6. Plates are read in the BioTek at OD_{530 nm}.
7. Plates are then placed in a plastic box which contains either a Petri dish of water. The box is sealed and incubated at 35° C. (Exophiala spp.) or 30° C. (Candida spp.). The presence of the water in the dish or on the tissue provides and environment of relatively high humidity.
8. The OD530 of the Candida spp. plates are read after 24 h and 48 h, while the slower growing Exophiala spp. plates are read after 96 h and 120 h.
9. Raw BioTek data is transformed using Gen5 software and represented graphically using GraphPad Prism 4 software and MICs determined.

### Plate Layouts

| Exophiala | FLUCONAZOLE | | | | | | ITRACONAZOLE | | | | | |
|---|---|---|---|---|---|---|---|---|---|---|---|---|
| Plate 1 | 1 | 2 | 3 | 4 | 5 | 6 | 7 | 8 | 9 | 10 | 11 | 12 |
| A | | 1000 | | | 3.91 | | | 32 | | | 0.125 | |
| B | | 500 | | | 1.95 | | | 16 | | | 0.063 | |
| C | | 250 | | | 0.98 | | | 8 | | | 0.031 | |
| D | | 125 | | | 0.49 | | | 4 | | | 0.016 | |
| E | | 62.5 | | | 0.24 | | | 2 | | | 0.008 | |
| F | | 31.25 | | | 0.12 | | | 1 | | | 0.004 | |
| G | | 15.63 | | | 0.06 | | | 0.5 | | | 0.002 | |
| H | | 7.81 | | | 0 | | | 0.25 | | | No Cells | |
| Candida | FLUCONAZOLE | | | | | | ITRACONAZOLE | | | | | |
| Plate 1 | 1 | 2 | 3 | 4 | 5 | 6 | 7 | 8 | 9 | 10 | 11 | 12 |
| A | | 500 | | | 1.95 | | | 32 | | | 0.125 | |
| B | | 250 | | | 0.98 | | | 16 | | | 0.063 | |
| C | | 125 | | | 0.49 | | | 8 | | | 0.031 | |
| D | | 62.5 | | | 0.24 | | | 4 | | | 0.016 | |
| E | | 31.25 | | | 0.12 | | | 2 | | | 0.008 | |
| F | | 15.63 | | | 0.06 | | | 1 | | | 0.004 | |
| G | | 7.81 | | | 0.03 | | | 0.5 | | | 0.002 | |
| H | | 3.91 | | | 0 | | | 0.25 | | | No Cells | |

| All Strains, | CASPOFUNGIN | | | | | | DMSO (%) | | | | | |
|---|---|---|---|---|---|---|---|---|---|---|---|---|
| Plate 2 | 1 | 2 | 3 | 4 | 5 | 6 | 7 | 8 | 9 | 10 | 11 | 12 |
| A | | 64 | | | 0.25 | | | 0.5 | | | 0.002 | |
| B | | 32 | | | 0.1 | | | 0.25 | | | 0.001 | |
| C | | 16 | | | 0.06 | | | 0.125 | | | 0.000 | |
| D | | 8 | | | 0.031 | | | 0.063 | | | 0.000 | |
| E | | 4 | | | 0.016 | | | 0.031 | | | 0.000 | |
| F | | 2 | | | 0.008 | | | 0.016 | | | 0.000 | |
| G | | 1 | | | 0.004 | | | 0.008 | | | 0.000 | |
| H | | 0.5 | | | 0 | | | 0.004 | | | No Cells | |

Antifungal Activity of Antifungal Drugs Used in Combination with Lynovex (NM001) Against Exophiala dermatitidis CA01.

### Aim:

The aim of the experiment was to determine the effect of Lynovex (NM001) on the antifungal drug susceptibility of Exophiala dermatitidis CA01. Microtitre plates were prepared following CLSI recommendations for broth microdilution chequerboard method. These plates contained a serial doubling dilution of Lynovex (NM001) combined with serial doubling dilutions of antifungals drugs; Fluconazole, Itraconazole, Caspofungin and Amphotericin B. Lynovex (NM001) and antifungals were initially prepared at 4× final concentration which were diluted to 2× final concentration following their combination. The cultures were then prepared in 2×RPMI-1640, and mixed in equal volumes in the plate resulting in 1×RPMI-1640, and the 1× concentration of compound mentioned below. These were incubated at 35° C. and read at 72 h, 96 h, 120 h and 144 h on the BioTek plate reader.

### Compounds for Antifungal Testing

Lynovex (NM001) in combination with Fluconazole, Itraconazole, Caspofungin and Amphotericin B

### Method:

### Culture Preparation

6. Three independent E. dermatitidis CA01 cultures were grown on PDA slope for 6 days at 35° C.
7. Cultures were removed from incubator. 2 ml sterile 0.15M NaCl added to the overnight culture and cells harvested from the surface of the agar. Cells placed in fresh bijou tube.
8. The cultures were allowed to rest for up to 10 minutes, allowing any hyphae to settle to the bottom. The top layer of conidia was removed into fresh bijou. Hyphae were discarded.
9. Two-fold serial dilutions carried out with each culture and optical density measured spectrophotometrically to attain the 0.5 McFarland Standard
   a. 100 μl culture added to F1 of microtitre plate.
   b. 100 μl sterile 0.15M NaCl added to wells F2-F12.
   c. 100 μl culture added to well F2 of microtitre plate and mixed by pipetting. This results in a 1 in 2 dilution.
   d. 100 μl taken from F2 and added to F3 on plate and mixed by pipetting. (1 in 4 dilution.)
   e. This is continued to F12 where 100 μl is removed and discarded.
   f. This dilution series was repeated in rows G-H for the remaining 2 cultures
   g. Plate read at OD₅₃₀ on BioTek
   h. Concentration of cells required will be the one that is the equivalent to the 0.5 McFarland Standard, or OD₅₃₀ at 0.12-0.15
10. Dilute cells to the equivalent of the 0.5 McFarland Standard (for example a 1 in 128 dilution may be required) in sterile 0.15M NaCl
11. Culture then further diluted 1 in 50 into 2×RPMI-1640 (1.1 ml culture+53.9 ml 2×RPMI-1640).

### Plate Preparation

This preparation step occurs in two parts. First, Lynovex (NM001) and antifungals are serially diluted, down and across microtitre plates, respectively. Equal volumes of Lynovex and antifungal are then combined in a fresh microtitre plate, prior to inoculation.

### Step 1: Antifungal Plate Preparation

6. Stock solutions of Lynovex and antifungals were prepared at 4× desired concentrations. The plate layout at the end of this protocol is the final concentration in the plate following the combination of Lynovex and antifungals and the addition of the inocula. For example, Lynovex is prepared initially at 10 mg/ml and the final concentration in the plate following addition of cells is 2.5 mg/ml.
7. 300 μl of Lynovex at 10 mg/ml is added to each well in row A.
8. All other wells contain 150 μl sterile dH₂O.
9. Serial doubling dilutions are carried out from A to B whereby 150 μl is removed from A and placed into B and mixed well. Then take 150 μl B and place in C and mix well. This is repeated down to row G where the final 150 μl is discarded. Row H contains water alone and will be antifungal only as well as untreated and uninoculated controls when combined.
10. The same procedure is repeated for the other 4 antifungals, carrying the dilutions across the plate i.e. Column 1 to 2. At the final dilution in column 10, the final 150 μl is discarded. Columns 11 and 12 contain water alone and will be Lynovex only as well as untreated and uninoculated controls when combined.

### Step 2: Challenge Plate Preparation

1. 50 μl Lynovex dilution from A1 combined with 50 μl antifungal dilution from A1 into A1 of fresh microtitre plate
2. This step is repeated until all wells have been combined, halving the Lynovex/antifungal concentration to a total volume of 100 μl.
3. Wells H11 and H12 contain 100 μl of sterile dH₂O and will become a no treatment and uninoculated control respectively.
   Inoculating Plates with E. dermatitidis CA01
6. Add 100 μl (total volume 200 μl) of the prepared E. dermatitidis CA01 culture to the all wells on the plate except wells H12. One culture was tested against all combinations, this yielded 3 replicates of each combination
7. H12 will contain 100 μl water and 100 μl 2×RPMI-1640 alone, constituting an uninoculated control.
8. The plate is read in the BioTek at OD₅₃₀.
9. Plates are then placed in a plastic box containing a Petri dish of water, sealed and incubated at 35° C. The presence of the water in the dish or on the tissue provides and environment of relatively high humidity.
10. OD₅₃₀ is then read at 72 h, 96 h, 120 h and 144 h and raw data processed using Microsoft Excel.
11. Fractional Inhibitory Concentration (FIC) Index values are generated using MIC₁₀₀ for 72 h data and MIC₅₀ for later time points using the following formula:

$$PICI = \begin{matrix}\begin{matrix}MIC Drug A in Combination \\ \text{?}\end{matrix} \\ MIC Drug A Alone\end{matrix} \text{?} \begin{matrix}\begin{matrix}MIC Drug B in Combination \\ \text{?}\end{matrix} \\ MIC Drug B Alone\end{matrix}$$

$$\text{?} \text{indicates text missing or illegible when filed}$$

### Plate Layout

### Step 1 Layouts:

| | 1 | 2 | 3 | 4 | 5 | 6 | 7 | 8 | 9 | 10 | 11 | 12 |
|---|---|---|---|---|---|---|---|---|---|---|---|---|
| A | 10,000 μg/ml Lynovex | | | | | | | | | | | |
| B | 5,000 μg/ml Lynovex | | | | | | | | | | | |
| C | 2,500 μg/ml Lynovex | | | | | | | | | | | |
| D | 1,250 μg/ml Lynovex | | | | | | | | | | | |
| E | 625 μg/ml Lynovex | | | | | | | | | | | |
| F | 312.5 μg/ml Lynovex | | | | | | | | | | | |
| G | 156.25 μg/ml Lynovex | | | | | | | | | | | |
| H | 0 μg/ml Lynovex | | | | | | | | | | | |
| A | χ | χ/2 | χ/4 | χ/8 | χ/16 | χ/32 | χ/64 | χ/128 | χ/256 | χ/512 | 0 | 0 |
| B | μg/ml | μg/ml | μg/ml | μg/ml | μg/ml | μg/ml | μg/ml | μg/ml | μg/ml | μg/ml | μg/ml | μg/ml |
| C | Anti- | Anti- | Anti- | Anti- | Anti- | Anti- | Anti- | Anti- | Anti- | Anti- | Anti- | Anti- |
| D | fungal | fungal | fungal | fungal | fungal | fungal | fungal | fungal | fungal | fungal | fungal | fungal |
| E | | | | | | | | | | | | |
| F | | | | | | | | | | | | |
| G | | | | | | | | | | | | |
| H | | | | | | | | | | | | |

Where χ is 120 μg/ml for Amphotericin B, 500 μg/ml for Caspofungin, 8000 μg/ml for Fluconazole and 64 μg/ml for Itraconazole.

### Step 2 Layouts:

| Antifungal (μg/ml) | | | | | | | | | | | |
|---|---|---|---|---|---|---|---|---|---|---|---|
| χ | χ/2 | χ/4 | χ/8 | χ/16 | χ/32 | χ/64 | χ/128 | χ/256 | χ/512 | 0 | 0 |
| 1 | 2 | 3 | 4 | 5 | 6 | 7 | 8 | 9 | 10 | 11 | 12 |

| | | |
|---|---|---|
| Lynovex | 2500 | A |
| (μg/ml) | 1250 | B |
| | 625 | C |
| | 312.5 | D |
| | 156.25 | E |
| | 78.125 | F |
| | 39.0625 | G |
| | 0 | H |

Where χ is 30 μg/ml for Amphotericin B, 125 μg/ml for Caspofungin, 2000 μg/ml for Fluconazole and 16 μg/ml for Itraconazole.

### Results

TABLE 1
In vitro minimum inhibitory concentrations (MICs) of Exophiala dermatitidis CA01 against the clinical
antifungals Fluconazole, Itraconazole and Caspofungin using CLSI M38-A2* broth microdilution procedure.
CLSI endorsed Candida spp. QC strains provide MIC values within the accepted range.

| (μg/ml) | | Fluconazole | | | Itraconazole | | | Caspofungin | | |
|---|---|---|---|---|---|---|---|---|---|---|
| Species | Strain | MIC₁₀₀ | MIC₈₀ | MIC₅₀ | MIC₁₀₀ | MIC₈₀ | MIC₅₀ | MIC₁₀₀ | MIC₈₀ | MIC₅₀ |
| Candida krusei | ATCC | 62.5 | 62.5 | 31.25 | 0.25 | 0.125 | 0.004 | 1 | 1 | 0.5 |
| | 6258 | | | | | | | | | |
| Candida parapsilosis | ATCC | 3.9 | 1 | 0.5 | 0.125 | 0.016 | 0.008 | 64 | 2 | 1 |
| | 22019 | | | | | | | | | |
| Exophiala jeanselmei | NCPF | 1000 | 500 | 250 | 0.25 | 0.25 | 0.125 | >64 | 64 | 0.125 |
| | 2377 | | | | | | | | | |
| Exophiala dermatitidis | CA01 | 500 | 500 | 250 | >32 | >32 | 2 | 32 | 32 | 16 |

*CLSI (2008) Reference Method for Broth Dilution Antifungal Susceptibility Testing of Filamentous Fungi; Approved Standard - Second Edition. (M38-A2).

TABLE 2
Broth microdilution chequerboard analysis of Lynovex (NM001) used in combination with
clinical antifugals. In vitro MIC₁₀₀ data of Lynovex and antifungals both in isolation
and in combination were generated following 72 h incubation at 35° C. and analysed
for FICI. FICI values for each combination were identified and averages are presented,
where <0.5; Synergy, 0.5-1; Additive, >1; Indifference. (Burkhart et al 2002)

| | | | NM001 | | | Antifungal | | |
|---|---|---|---|---|---|---|---|---|
| | | | MIC₁₀₀ in | | | MIC₁₀₀ in | | |
| | | NM001 | Combination | | Antifungal | Combination | | FICI |
| ANTIFUNGAL | STAT | MIC₅₀ | (n) | | MIC₁₀₀ | (n) | | (n) |
| Itraconazole | MEAN | 1510 | 203 | | 4.7 | 0.24 | | 0.19 (5) |
| | GEOMEAN | 1250 | 179 | | 4.5 | 0.14 | | 0.18 (5) |
| | Range | 312.5-2500 | 78-312.5 | (5) | 4.0-8.0 | 0.03-0.5 | (5) | |
| Fluconazole | MEAN | 1250 | 365 | | 250 | 34 | | 0.43 (3) |
| | GEOMEAN | 1250 | 312.5 | | 250 | 25 | | 0.42 (3) |
| | Range | 1250 | 156-625 | (3) | 250 | 7.81-62.5 | (3) | |
| Amphotericin B | MEAN | 667 | 39 | | 0.86 | 0.23 | | 0.53 (1) |
| | GEOMEAN | 461 | 39 | | 0.59 | 0.23 | | 0.53 (1) |
| | Range | 125-1250 | 39 | (1) | 0.23-1.88 | 0.23 | (1) | |
| Caspofungin | MEAN | 1458 | 78 | | 26 | 2.0 | | 0.25 (1) |
| | GEOMEAN | 1250 | 78 | | 25 | 2.0 | | 0.25 (1) |
| | Range | 625-2500 | 78 | (1) | 15.625-31.25 | 2.0 | (1) | |

TABLE 3
Broth microdilution chequerboard analysis of Lynovex (NM001) used in combination with
clinical antifugals. In vitro MIC₅₀ data of Lynovex and antifungals both in isolation
and in combination were generated following 96 h incubation at 35° C. and analysed
for FICI. FICI values for each combination were identified and averages are presented,
where <0.5; Synergy, 0.5-1; Additive, >1; Indifference. (Burkhart et al 2002)

| | | | NM001 | | | Antifungal | | |
|---|---|---|---|---|---|---|---|---|
| | | | MIC₅₀ in | | | MIC₅₀ in | | |
| | | NM001 | Combination | | Antifungal | Combination | | |
| ANTIFUNGAL | STAT | MIC₅₀ | (n) | | MIC₅₀ | (n) | | FICI (n) |
| Itraconazole | MEAN | 1518 | 89 | | 4.0 | 0.74 | | 0.32 (7) |
| | GEOMEAN | 1132 | 78 | | 4.0 | 0.37 | | 0.27 (7) |
| | Range | 312.5-2500 | 39.06-156.25 | (7) | 4.0 | 0.06-2 | (7) | |
| Fluconazole | MEAN | 885 | 176 | | 229 | 51 | | 0.45 (6) |
| | GEOMEAN | 787 | 139 | | 223 | 35 | | 0.44 (6) |
| | Range | 312.5-1250 | 39.06-156.25 | (6) | 125-250 | 7.81-125 | (6) | |
| Amphotericin B | MEAN | 885 | 59 | | 0.47 | 0.18 | | 0.64 (2) |
| | GEOMEAN | 625 | 55 | | 0.47 | 0.17 | | 0.63 (2) |
| | Range | 156.25-1250 | 39.0625-78.125 | (2) | 0.47 | 0.12-0.23 | (2) | |
| Caspofungin | MEAN | 1531 | 102 | | 31 | 14.84 | | 0.58 (4) |
| | GEOMEAN | 1088 | 90 | | 31 | 11.84 | | 0.48 (4) |
| | Range | 156.25-2500 | 39.0625-156.25 | (4) | 31.25 | 3.91-31.25 | (4) | |

TABLE 4
Broth microdilution chequerboard analysis of Lynovex (NM001) used in combination with clinical antifugals.
In vitro MIC₅₀ data of Lynovex and antifungals both in isolation and in combination were generated following
120 h incubation at 35° C. and analysed for FICI. FICI values for each combination were identified and
averages are presented, where <0.5; Synergy, 0.5-1; Additive, >1; Indifference. (Burkhart et al 2002)

| | | | NM001 | | | Antifungal | | |
|---|---|---|---|---|---|---|---|---|
| | | | MIC₅₀ in | | | MIC₅₀ in | | |
| | | NM001 | Combination | | Antifungal | Combination | | |
| ANTIFUNGAL | STAT | MIC₅₀ | (n) | | MIC₅₀ | (n) | | FICI (n) |
| Itraconazole | MEAN | 1500 | 156.25 | | 4.8 | 0.33 | | 0.20 (5) |
| | GEOMEAN | 1250 | 136 | | 4.6 | 0.19 | | 0.18 (5) |
| | Range | 625-2500 | 78.13-312.5 | (5) | 4-8 | 0.06-1 | (5) | |
| Fluconazole | MEAN | 1875 | 312.5 | | 219 | 59 | | 0.42 (4) |
| | GEOMEAN | 1768 | 263 | | 210 | 44 | | 0.41 (4) |
| | Range | 1250-2500 | 156.25-625 | (4) | 125-250 | 15.63-125 | (4) | |
| Amphotericin B | MEAN | 977 | 221 | | 0.47 | 0.20 | | 0.59 (3) |
| | GEOMEAN | 743 | 156 | | 0.47 | 0.19 | | 0.58 (3) |
| | Range | 156.25-1250 | 39.06-312.5 | (3) | 0.5 | 0.12-0.23 | (3) | |
| Caspofungin | MEAN | 1750 | 148 | | 22 | 4.20 | | 0.27 (4) |
| | GEOMEAN | 1436 | 118 | | 21 | 1.95 | | 0.24 (4) |
| | Range | 625-2500 | 39.06-312.5 | (4) | 15.63-31.25 | 0.49-15.625 | (4) | |

TABLE 5
Broth microdilution chequerboard analysis of Lynovex (NM001) used in combination with
clinical antifugals. In vitro MIC₅₀ data of Lynovex and antifungals both in isolation
and in combination were generated following 144 h incubation at 35° C. and analysed
for FICI. FICI values for each combination were identified and averages are presented,
where <0.5; Synergy, 0.5-1; Additive, >1; Indifference. (Burkhart et al 2002)

| | | | NM001 | | | Antifungal | | |
|---|---|---|---|---|---|---|---|---|
| | | | MIC₅₀ in | | | MIC₅₀ in | | |
| | | NM001 | Combination | | Antifungal | Combination | | |
| ANTIFUNGAL | STAT | MIC₅₀ | (n) | | MIC₅₀ | (n) | | FICI (n) |
| Itraconazole | MEAN | 2031 | 208 | | 8 | 0.27 | | 0.19 (3) |
| | GEOMEAN | 1768 | 197 | | 6.7 | 0.20 | | 0.18 (3) |
| | Range | 625-2500 | 156.25-312.5 | (3) | 4->16 | 0.06-0.5 | (3) | |
| Fluconazole | MEAN | 1719 | 400 | | 375 | 98 | | 0.55 (4) |
| | GEOMEAN | 1487 | 263 | | 354 | 63 | | 0.53 (4) |
| | Range | 625-2500 | 39.0625-625 | (4) | 250-500 | 15.63-250 | (4) | |
| Amphotericin B | MEAN | 1302 | 469 | | 0.63 | 0.23 | | 0.75 (2) |
| | GEOMEAN | 787 | 442 | | 0.59 | 0.23 | | 0.75 (2) |
| | Range | 156.25-2500 | 312.5-625 | (2) | 0.47-0.94 | 0.23 | (2) | |
| Caspofungin | MEAN | 1875 | 417 | | 26 | 6.51 | | 0.52 (3) |
| | GEOMEAN | 1575 | 394 | | 25 | 3.91 | | 0.41 (3) |
| | Range | 625-2500 | 312.5-625 | (3) | 0.47-0.94 | 0.23 | (3) | |

1. A composition comprising cysteamine or a derivative thereof for use in the treatment or prevention of an infection caused by yeasts and/or moulds.

2. A composition according to claim 1 wherein the infection is caused by one or more of the group consisting of: Candida spp., (e.g. C. albicans), Aspergillus spp., Epidermophyton spp., Exophiala spp., Microsporum spp., Trichophyton spp., (e.g T. rubrum and T. interdigitale), Tinea spp., Blastomyces spp., Blastoschizomyces spp., Coccidioides spp., Cryptococcus spp. (e.g. Cryptococcus neoformans), Histoplasma spp., Paracoccidiomyces spp., Sporotrix spp., Absidia spp., Cladophialophora spp., Fonsecaea spp., Phialophora spp., Lacazia spp., Arthrographis spp., Acremonium spp., Actinomadura spp., Apophysomyces spp., Emmonsia spp., Basidiobolus spp., Beauveria spp., Chrysosporium spp., Conidiobolus spp., Cunninghamella spp., Fusarium spp., Geotrichum spp., Graphium spp., Leptosphaeria spp., Malassezia spp. (e.g Malassezia furfur), Mucor spp., Neotestudina spp., Nocardia spp., Nocardiopsis spp., Paecilomyces spp., Phoma spp., Piedraia spp., Pneumocystis spp., Pseudallescheria spp., Pyrenochaeta spp., Rhizomucor spp., Rhizopus spp., Rhodotorula spp., Saccharomyces spp., Scedosporium spp., Scopulariopsis spp., Sporobolomyces spp., Syncephalastrum spp., Trichoderma spp., Trichosporon spp., Ulocladium spp., Ustilago spp., Verticillium spp., Wangiella spp.

3. A composition according to claim 1 wherein the infection is caused by Candida spp., Aspergillus spp. or Cryptococcus spp.

4. A composition according to claim 1 wherein the infection is caused by Exophiala spp.

5. A composition comprising cysteamine for use in the prevention or treatment of any one or more of the group consisting of: candidiasis (including OPC), aspergillosis (including bronchopulmonary aspergillosis, chronic pulmonary aspergillosis and aspergillomata), cystic fibrosis, athlete's foot; basidiodiabolomycosis; blastomycosis; coccidioidomycosis cryptoccocis; COPD; basal meningitis; dermatophytosis; onchomycosis; dermatophytids; endothrix; exothrix; fungal meningitis, fungemia, heaves; histoplasmosis, mycosis, myrinogmycosis, paracoccidioidomycosis, penicilliosis, piedra, pneumocytosis pneumonia, sporptrichosis, tinea, zeospora and zygomycosis.

6. A composition according to claim 1, wherein said composition is a pharmaceutical composition comprising a pharmaceutically acceptable carrier, excipient or diluent

7. A composition according to claim 1, wherein the composition comprises an antibiotic or an additional antifungal.

8. A composition according to claim 1, wherein the antifungal is selected from one or more of the group consisting of: Fluconazole, Itraconazole, Caspofungin and Amphotericin B.

9. A method of treating or preventing an infection caused by yeasts or moulds in a subject comprising administering a pharmaceutically effective amount of a composition comprising cysteamine or a derivative thereof.
